(19) United States
(12) Patent Application Publication
(10) Pub. No.: US 2016/0193073 A1
(43) Pub. Date: Jul. 7, 2016

(54) CATHETER SUPPORT SYSTEMS AND METHODS OF USE

(71) Applicants: Gary Kinsey, Gainesville, FL (US); Frank Pupello, LandLakes, FL (US); Cynthia Harris, Gainesville, FL (US); Gefrey Humphries, Obrien, FL (US)

(72) Inventors: Gary Kinsey, Gainesville, FL (US); Frank Pupello, LandLakes, FL (US); Cynthia Harris, Gainesville, FL (US); Gefrey Humphries, Obrien, FL (US)

(21) Appl. No.: 14/986,927

(22) Filed: Jan. 4, 2016

Related U.S. Application Data
(60) Provisional application No. 62/099,434, filed on Jan. 2, 2015.

(51) Int. Cl. A61F 5/44; F16L 3/02; A61M 39/08

(52) U.S. Cl. CPC A61F 5/4408; A61M 39/08; F16L 3/02

(57) ABSTRACT

The present invention provides for a disposable devices and kits to prevent the backflow of urine and other biofluids from a catheter bag and tubing into a patients bladder and further provides for the appropriate management of a catheter bag and tubing during the transport of a patient, while reducing the risk of a urinary tract infection (UTI) and maintenance of dignity. The advantages of the present invention make use of an inventive tubing retainer that is employed for catheter tube management. The tubing retainer may alternatively be included in a kit used for the management and transport of a catheter bag and flexible tubing which further includes a clip and a strap used to connect between the clip and the catheter and alternatively providing support of the catheter tube at the catheter inlet. Methods to use the tubing retainer and kits are further described.

### CROSS-REFERENCE TO RELATED APPLICATIONS

The present application claims priority of U.S. Provisional patent application No. 62/099,434 filed Jan. 2, 2015, the contents of which are herein incorporated by reference.

### FIELD OF THE INVENTION

The present invention relates to medical devices, and more particularly, to a catheter and catheter tubing management kit and its method of use.

### BACKGROUND OF THE INVENTION

Urinary tract infections (UTI) are becoming a regular occurrence in the medical setting. Whether in hospitals, urgent care centers, outpatient centers, primary care providers, and even in retirement homes and hospice facilities, typically any patient who receives a catheter encounters a high risk of receiving a UTI. Once a catheter is placed, the daily incidence of a UTI is 3-10%. 10% to 30% of patients who undergo short-term catheterization (i.e. 2-4 days) develop an UTI. Between 90% and 100% of patients who undergo long-term catheterization develop UTI. Although UTI's are relatively easy to treat, they increase patient discomfort and almost universally require antibiotic drug therapy. These additional treatments and procedures ultimately lead to unnecessary costs in an industry where increased costs are being widely scrutinized.

UTI's occur in medical settings for many reasons. Risk factors for a UTI in patients who are catheterized include: patient's lack of hygiene, improper sterility of catheter tubing during extended catheterization, longer duration of catheterization, lack of proper drainage and subsequent urine backflow, excessive manipulation of the catheter, catheter tubing, and its attendant drainage bag during patient transport, colonization of the drainage bag, diarrhea, diabetes, absence of antibiotics, female gender, renal insufficiency, errors in catheter care, catheterization late in the hospital course, and immunocompromised or debilitated states.

Without being bound by any particular theory, it is believed that a common cause of UTI in the medical setting is the failure to have proper tubing and drainage bag placement, thus urine and other biofluids in the catheter tubing make their way back to the bladder, thus causing infection. Current solutions are to simply advise medical staff to ensure proper drainage by keeping the tubing and drainage bag at an elevation below the patient's bladder while the tubing remains above the drainage bag. Currently these are only “best practices” and no device or kit has been offered to ensure adherence to these “best practices”.

In any event, and despite the “best practices”, transportation of a patient presents a challenge at ensuring these “best practices” can remain employed for a patient. Not only is it difficult to maintain the proper relative elevations of the bag and tubing, but the tubing and bag also increase the chances of tangle and snagging on other items while in transport which could cause movement of the catheter, increasing the likelihood of infection. Moreover, a patient is usually required to carry or place the drainage bag on their lap, or to dangle the bag and tubing from a chair or rollable tracks or carriers which is typically unsightly and embarrassing for the patient. Finally, the few solutions provided to address any concerns with catheters are typically very costly and not disposable, thus are rarely used due to the extra costs associated with recycling and sterilizing the accessories.

No kit or device has been offered to date to account for any of these shortcomings, thus there remains an unmet need for a disposable device or kit to assist in preventing the backflow of urine and other biofluids from a catheter bag and tubing in to a patients bladder. There also remains an unmet need in a kit or device for the management of a catheter bag and tubing while transporting a patient, wherever the patient may be required to go, such as MRI/CT scan platforms or rehab. Finally, there remains an unmet need in transporting a patient and his catheter while reducing the risk of a UTI, while allowing the patient to keep their dignity in the transportation of their catheter.

### SUMMARY OF INVENTION

The present invention provides for a disposable devices and kits to prevent the backflow of urine and other biofluids from a catheter bag and tubing into a patients bladder and further provides for the appropriate management of a catheter bag and tubing during the transport of a patient, while reducing the risk of a urinary tract infection (UTI) and maintenance of dignity.

The present invention first includes a tubing retainer having a bracket with two sections defined as a tube management section and a retainer storing section. The tubing management section and retainer storing section each have at least one hole or partial circle forming a c-shape for holding a tube, but in some embodiments may have a plurality of holes of partial circles.

The disposable kits of the present invention provide for the management and transport of a catheter bag and flexible tubing, and include the inventive tubing retainer, at least one strap having two ends, a first end and a second end, and at least one clip wherein said at least one clip is connected to the first end of the at least one strap. The strap may include a plurality of independent or composite straps that include a long strap and a short strap having two ends, a first end and a second end, with the long strap being used to connect between at least one clip and the catheter, while the short strap may be used for tubing management or tubing management or support at the catheter inlet.

Finally, the present invention provides methods employed using the inventive tubing retainer and kits for the management of a catheter system attached to a patient having a catheter and catheter tube.

### BRIEF DESCRIPTION OF THE DRAWINGS

FIGS. 1A-1C illustrates a 102 strap clip which assists in the management of a catheter bag and tubing. FIG. 1A provides a side profile view illustrating the 102 strap clip having a 201 distal end and a 202 proximal end with a 204 slight bend at a distance from the 201 distal end and a 203 major bend at some distance between said 204 slight bend and said 202 proximal end. FIG. 1B illustrates a perspective view of the 102 strap clip illustrating a 205 strap eyelet at a distance between said 201 distal end and said 204 slight bend. FIG. 1C provides a front view illustrating the 102 strap clip.

FIGS. 2A-2B illustrates a 101 tubing retainer for holding coiled flexible catheter tubing. FIG. 2A provides a top view of the 101 tubing retainer illustrating the 301 tubing management section and the 302 retainer storing section each having at least one 303 hole. FIG. 2B provides a flat side view of the 101 tubing retainer illustrating the orientation of the 303 holes.

FIGS. 3A-3C illustrate the 100 catheter support kit and its use. FIG. 3A introduces the 103 strap which in some embodiments has one or more 304 adhesive pads. FIG. 3B illustrates the constituents of the 100 catheter support kit which includes the 101 tubing retainer, the 102 strap clip and the 103 strap. FIG. 3C illustrates the use of the 100 catheter support kit on a catheter showing the 101 tubing retainer coiling the 402 catheter tubing to provide a trap or dependent loop, while showing the 103 strap connected between the 401 catheter and the 102 strap clip.

FIG. 4 illustrates the 101 tubing retainer coiling the 402 catheter tubing to provide a trap or dependent loop.

FIGS. 5A-5C show uses of the inventive 100 catheter support kit. FIG. 5A illustrates the 100 kit in use (as shown in FIG. 3C) with the 102 strap clip hanging from the back of a wheel chair. FIG. 5B illustrates the 100 kit in use (as shown in FIG. 3C) with the 102 strap clip hanging from the belt of an individual. FIG. 5C illustrates the 100 kit in use (as shown in FIG. 3C) being attached to the side rails of a hospital bed. Additionally, FIG. 5C illustrates that the 103 strap may be used to form a loop around the bed rail in the event that the 102 strap clip cannot accommodate the size of an object to be hung from.

### DETAILED DESCRIPTION OF THE INVENTION

The following detailed description is merely exemplary in nature and is in no way intended to limit the scope of the invention, its application, or uses, which may vary. The invention is described with relation to the non-limiting definitions and terminology included herein. These definitions and terminology are not designed to function as a limitation on the scope or practice of the invention, but are presented for illustrative and descriptive purposes only.

The present invention contains the features of the urinary catheter management system and methods of use as herein described.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4

### Tubing Retainer

A 101 tubing retainer for holding coiled flexible catheter tubing is provided which includes a bracket having two sections, a 301 tube management section and a 302 retainer storing section. The 301 tubing management section and the 302 retainer storing section each have at least one 303 hole for holding a tube. The 301 tubing management section is intended for holding in place coiled tubes for proper urine flow, while the 302 retainer storing section is used for optionally holding a section of catheter tube when the tubing management section is in use or is not in use. Without being bound by any particular theory, the 101 tubing retainer is intended to assist in creating a continuous flow for the urine and other biological fluids to the catheter bag, while creating a trap, or dependent loops, preventing the biological fluids from travelling back up the tubing even in the event that the recommended relative elevations of the catheter bag and tubing, in relation to the patients bladder, is not maintained or is disturbed as a result of transporting the patient.

In at least one embodiment, the 303 at least one hole in the 301 tubing management section and the 302 retainer storing section is a partial circle forming a c-shape. In at least one embodiment the 303 c-shaped hole has two indents slightly protruding the diameter of the hole. In at least one embodiment, the 301 tubing management section has at least three 303 holes.

It is appreciated that many suitable materials are known in the art for the construction of disposable medical devices, and nothing herein is intended to be limiting the 101 tubing retainer to any particular material. In at least one embodiment, the 101 tubing retainer is made from plastic, glass reinforced plastic, fiberglass, metal, wood, poly vinyl chloride (PVC), polycarbonate material, silicon, stainless steel, aluminum or combinations thereof. In at least one embodiment the materials selected is intended to be disposable, while structurally sound for providing the functionality described herein. In at least one embodiment the 101 tubing retainer is made from PVC or a polycarbonate material.

### Strap Clip

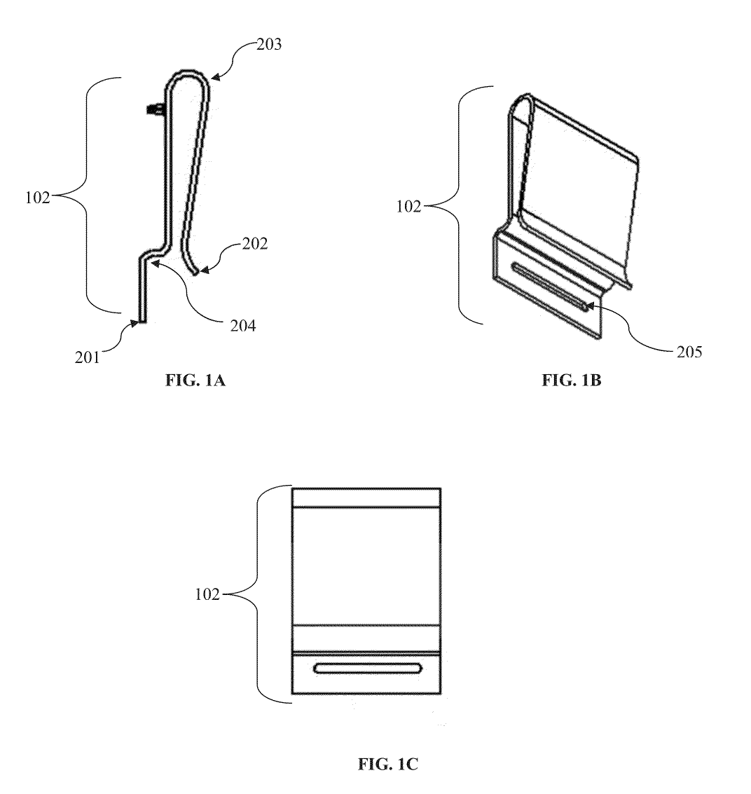

A 102 strap clip for suspending a catheter bag from an object is provided which includes a rectangular plate that forms a “U” shape clip having a 201 distal end and a 202 proximal end and is illustrated in FIGS. 1A-1C. The 102 strap clip has at least one 205 strap eyelet for receiving one end of a strap. The other end of the strap is connected to a catheter bag. The 102 strap clip allows for the ease of transportation of a catheter bag by holding the bag in a position not likely to get tangled, and based on placement, will assist to ensure that the catheter bag will remain below the bladder of the patient. The 102 strap clip may be connected to the back or seat of a chair or wheelchair, or may be connected to the belt or belt loop of the patient, medical staff assistant, or any other person or object who may be assisting the patient. The 102 strap clip includes a 204 slight bend at a distance from the 201 distal end and a 203 major bend at some distance between said 204 slight bend and said 202 proximal end, thus the 102 strap clip generally forming a “U” shape. In at least one embodiment the 204 slight bend is at least a 15 degree bend.

In at least one embodiment the 204 slight bend is at least a 45 degree bend. In at least one embodiment, the 204 slight bend is at least a 90 degree bend with the bend terminating where the direction of the 102 clip continues to travel away from the 201 distal end. It should be appreciated that two or more bends may be used to attain the 204 slight bend. In at least one embodiment the 203 major bend is at least 90 degrees. In at least one embodiment the 203 major bend is at least 180 degrees with the bend terminating where the direction of the 102 clip continues to travel back in the same direction as the 201 distal end. The 205 strap eyelet is located some distance between the 201 distal end and the 204 slight bend.

It is appreciated that many suitable materials are known in the art for the construction of disposable medical devices, and nothing herein is intended to be limiting the 102 strap clip to any particular material. In at least one embodiment, the 102 strap clip is made from plastic, glass reinforced plastic, fiberglass, metal, wood, poly vinyl chloride (PVC), silicon, stainless steel, aluminum or combinations thereof. In at least one embodiment the materials selected is intended to be disposable, while structurally sound for providing the functionality described herein. In at least one embodiment the strap clip is made from PVC.

### Kit

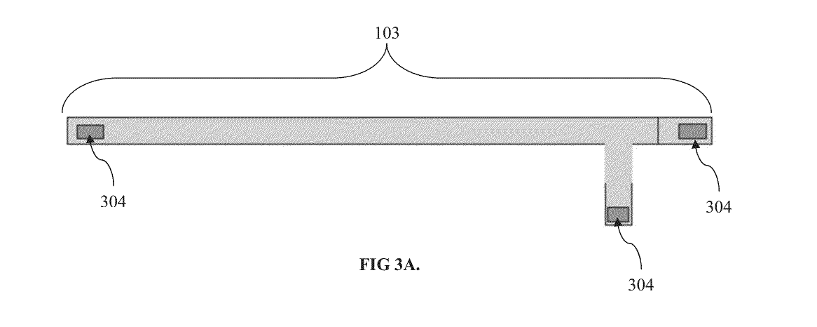

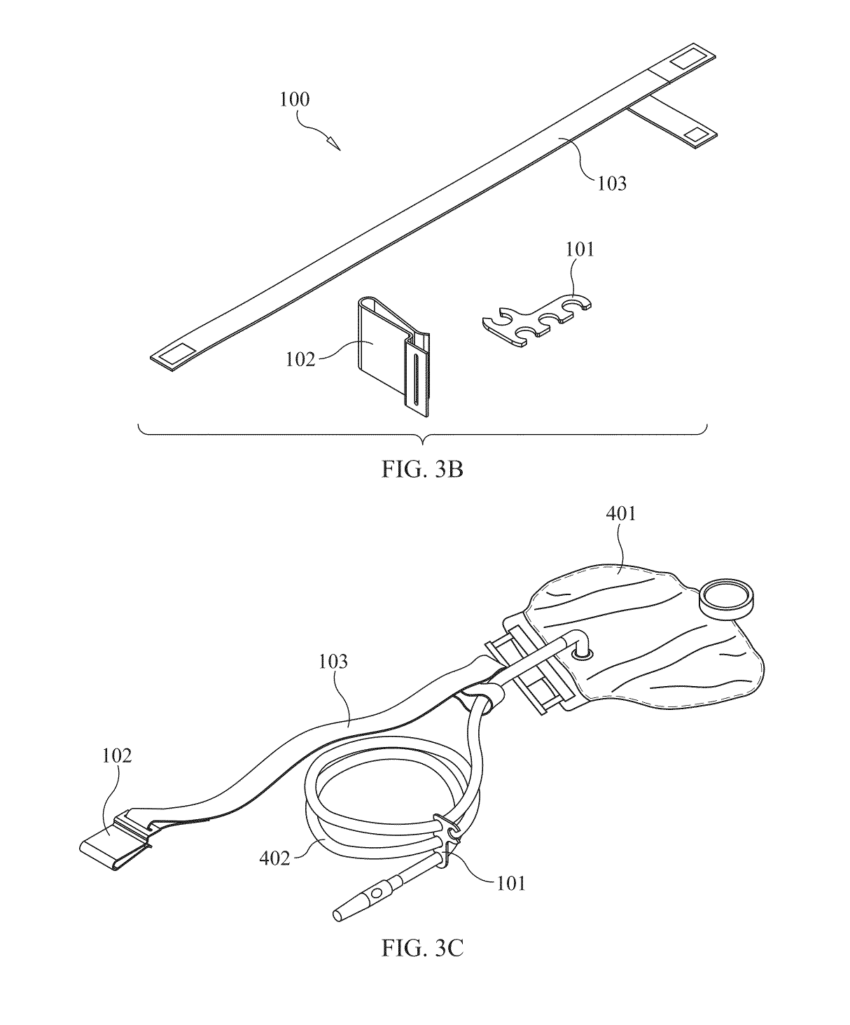

A disposable kit for the management and transport of a catheter bag and flexible tubing, is provided which includes at least one 101 tubing retainer, at least one 102 strap clip and at least one 103 strap having a first end and a second end. The first end of the 103 strap is connected to the strap clip while the second end of the 103 strap is for the connection to a catheter bag. It is appreciated that the 103 strap may include one or more 304 adhesive tabs. The 304 adhesive tabs may be used to shorten the length of the 103 strap, or to form loops on the strap for connecting to a 102 strap clip or a catheter. In some embodiments the 304 strap may comprise of a combination of a short strap and a long strap as illustrated in FIG. 3A. The short strap and long strap may come as two separate pieces, or may be part of one 304 strap. The long strap, where used alone or in combination with the short strap, is at least 6 inches in length having two ends, a first end and a second end. In at least one embodiment the short strap is at least 1 inch in length, and no greater than 6 inches in length having two ends, a first end and a second end.

The strap allows or the connection of the catheter bag and tubing to many movable objects, including humans, thus allowing gravity to maintain the catheter bag and tubing below the proximate location of the patients bladder. In at least one embodiment the strap has an adjustable length between 6 inches and 3 feet. In at least one embodiment the strap has an adjustable length between 1 to 2 feet. It is appreciated that many suitable materials are known in the art for the construction of disposable medical devices and lanyards, and nothing herein is intended to be limiting the strap to any particular material. In at least one embodiment, the strap is made from nylon, rubber, hook and loop fasteners, glass fiber, burlap, Tyvek, cotton, polyester, or combinations thereof. In at least one embodiment the materials selected is intended to be disposable, while structurally sound for providing the functionality described herein. In at least one embodiment the strap is made from Tyvek.

It is appreciated that many catheter bags have hooks for holding or storing the catheter bag pre-fabricated into their structure. In at least one embodiment the strap further comprises a loop at said second end for holding the hook of a catheter strap.

In at least one embodiment the kit further includes a dignity bag, or a bag for holding and disguising a catheter bag. It is appreciated that many suitable materials are known in the art for the construction of disposable medical bags, and nothing herein is intended to be limiting the dignity bag to any particular material. In at least one embodiment, the dignity bag is made from nylon, rubber, glass fiber, plastic, silicon, burlap, Tyvek, cotton, polyester, or combinations thereof.

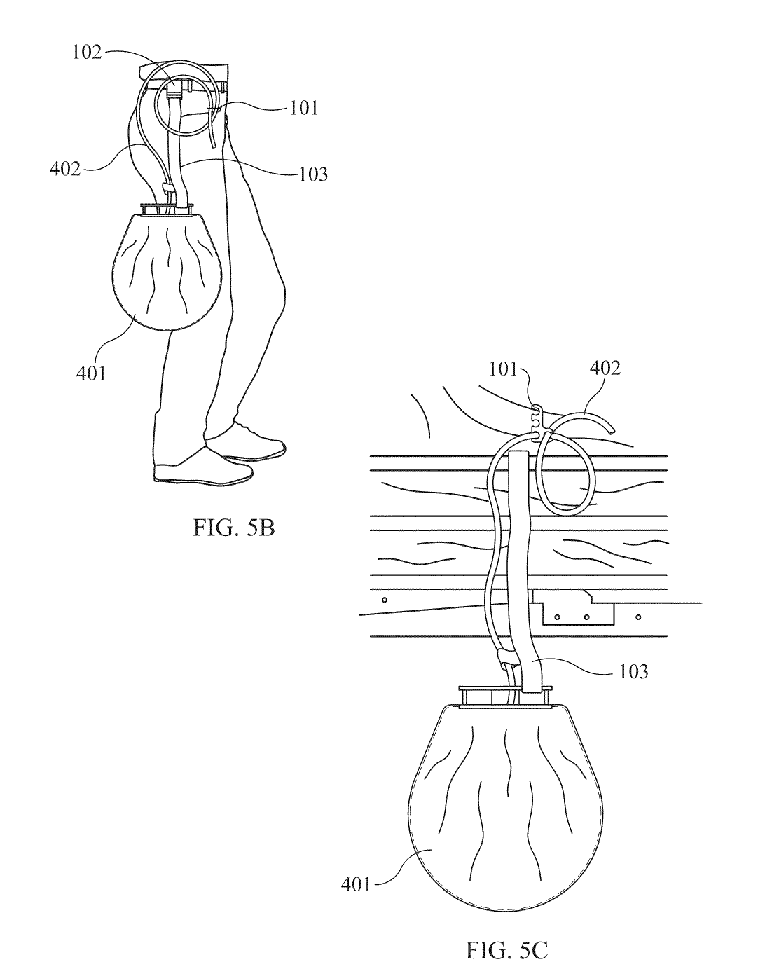

Finally, the kit further includes printed instructions for attaching the first end of the strap to the 102 strap clip, the second end of the strap to the catheter bag, and instructions for coiling the catheter tubing and attaching the 101 tubing retainer to the coiled catheter tubing. It should be appreciated that the instructions provided are not required to be in any specific order, and the numbering of such printed instructions shall be read to require the performance of one step prior to the performance of any other step. It is further appreciated that the steps may be performed in any order with the goal of decreasing the amount of pain on the patient and/or facilitating the maintenance of the elevation of the catheter bag below the bladder of the patient. Uses of the kit in relation to different objects are illustrated in FIGS. 5A-5C.

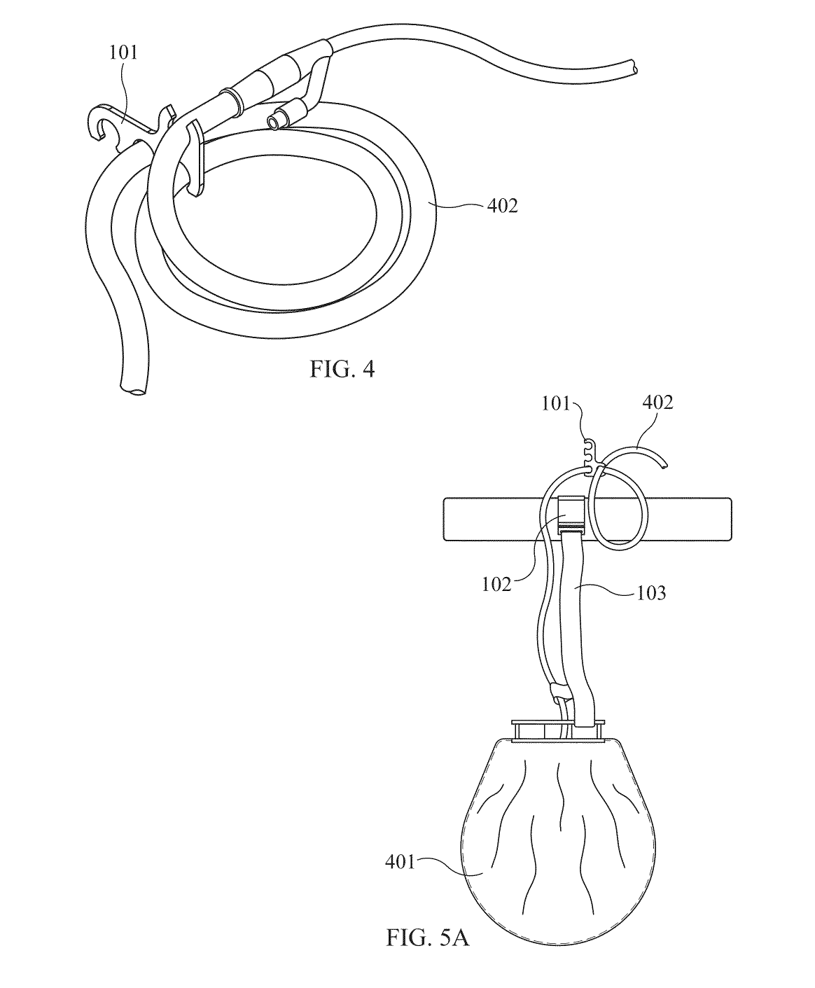

The drawings and diagrams shown herein depict example arrangements of elements of the kit, its individual components and the methods described herein. More or less than all the features available or contemplated may be present in an actual embodiment. It should also be understood that FIGS. 1A-1C, 2A-2B, 3A-3C, 4, & 5A-5C are merely illustrative and may not be drawn to scale.

### EXAMPLES

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### Example 1

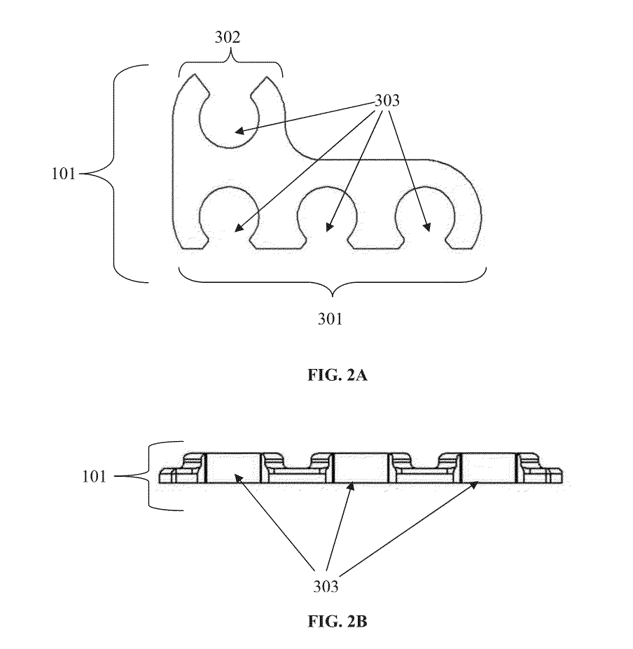

A 100 kit for the management of a 401 catheter bag and 402 tubing is provided to assist in the transportation of a patient, with the kit as illustrated in FIG. 3A and FIG. 3B. The kit includes a 101 tubing retainer, a 102 strap clip, and a 103 strap. The 101 tubing retainer has three 303 holes forming a c-shape on the 301 tubing management section and one 303 hole forming a c-shape on the 302 retainer storing section. The 101 tubing retainer is provided and illustrated in FIGS. 2A-2B. The 102 strap clip has a 204 slight bend of approximately 90 degrees, and a 203 major bend of approximately 180 degrees. The 103 strap provided is a Tyvek strap of approximately 16 inches in length, having 304 adhesive at each end for securing the strap or forming loops for holding a catheter bag, as necessary. The 100 kit further includes printed instruction for attaching the 103 strap to the 102 strap clip, the 103 strap to the 401 catheter bag, and the 101 tubing retainer to the 402 catheter tubing.

### Example 2

The 100 kit from Example 1 is provided for the management of a catheter system attached to a patient. The catheter system includes a 401 catheter bag and 402 tubing. A nurse removes the contents of the 100 kit and proceeds to follow the written instructions. The nurse decides first where to attach the strap for the transport of the patient, which in this case the nurse intends to attach the strap to the waist of the nurse for tandem transit with the patient (see FIG. 5B). Next, the nurse connects the first end of the 103 strap to the 102 strap clip by sliding the 103 strap through the 205 strap eyelet, then exposing the 304 adhesive on the first end to form a loop and securing the 102 strap clip into the loop formed by the 103 strap. Next the nurse forms a loop at the second end of the 103 strap by exposing the 304 adhesive at the second end of the 103 strap and forming a loop for holding the hook of the 401 catheter bag. The nurse adjusts the strap length before securing the 304 adhesive such that the 401 bag and 402 tubing will remain below the patents bladder while transporting. The patient is moved from their gurney to a wheelchair. The nurse coils the 402 catheter tube and uses the 101 tubing retainer for retaining the coiled 402 catheter tubing. The nurse supports the 102 strap clip with their hand, while attaching the hook of the 401 catheter bag to the loop formed at the second end of the 103 strap. The 102 strap clip is alternatively attached to the patient's chair (see FIG. 5A), the patient (see FIG. 5C), or to some other movable object, provided that the catheter bag and tubing remain below the patient's bladder.

After the transportation is complete, or upon the removal of the catheter of the patient, the medical staff handing the discarded 401 catheter bag disposes of the 101 tubing retainer, the 102 strap clip, and the 103 strap are discarded in the appropriate waste receptacle.

### Other Embodiments

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient road map for implementing the exemplary embodiment or exemplary embodiments. It should be understood that various changes can be made in the function and arrangement of elements without departing from the scope as set forth in the appended claims and the legal equivalents thereof.

1. A tubing retainer comprising:
a bracket having two sections;
a tube management section; and
a retainer storing section.

2. The tubing retainer of claim 1 wherein said tubing management section and said retainer storing section each have at least one hole for holding a tube.

3. The tubing retainer of claim 2 wherein said at least one hole is a partial circle forming a c-shape in said tubing management section and said retainer storing section.

4. The tubing retainer of claim 3 wherein said c-shaped hole has two indents slightly protruding the diameter of the hole.

5. The tubing retainer of claim 1 wherein said tubing management section has at least 3 holes.

6. The tubing retainer of claim 1 wherein said retainer is made of plastic, glass reinforced plastic, fiberglass, metal, wood, poly vinyl chloride (PVC), silicon, or combinations thereof.

7. A disposable kit for the management and transport of a catheter bag and flexible tubing, the kit comprising:
the tubing retainer of claim 1;
at least one long strap having two ends, a first end and a second end;
at least one short strap having two ends, a first end and a second end; and
at least one clip;
wherein said at least one clip is connected to the first end of the at least one long strap.

8. The kit of claim 7 wherein said long strap or said short strap is made of a disposable material.

9. The kit of claim 7 wherein said long strap or said short strap is made from nylon, rubber, glass fiber, burlap, Tyvek, cotton, polyester, or combinations thereof.

10. The kit of claim 7 wherein said long strap further comprises a loop at said second end.

11. The kit of claim 7 further comprising a dignity bag for holding and disguising a catheter bag, wherein said dignity bag is made from nylon, rubber, glass fiber, burlap, Tyvek, cotton, polyester, or combinations thereof.

12. The kit of claim 7 wherein said clip is made from a polyvinyl chloride (PVC), polycarbonate, plastic, wood, metal, ceramics, or combinations thereof.

13. The kit of claim 7 wherein said short strap is attached perpendicularly to said long strap at the second end to act as a tubing support strap at catheter inlet tubing.

14. A method for the management of a catheter system attached to a patient having a catheter and catheter tube, the method comprising:
obtaining at least one kit of claim 7;
connecting said clip to said first end of said strap;
attaching a catheter to said second end of said strap;
coiling the catheter tube;
retaining the coiled catheter tube using said tubing retainer;
attaching said clip holding the catheter by the strap to a movable object to accompany said patient.

15. The method of claim 14 further comprising, forming a loop with said second end of said strap for receiving a hook from a catheter bag.

16. The method of claim 14 wherein the catheter tube is coiled at least three times to prevent backflow of biological materials from said catheter bag to the patient through the catheter tube.

17. The method of claim 14 further comprising inserting said catheter bag into a dignity bag to obscure the contents of said catheter bag.

18. The method of claim 14 wherein said short strap is attached perpendicularly to said long strap at the second end of said long strap, and wrapped around the catheter tubing near where the catheter tubing mates with the catheter bag, to act as a tubing support strap at the catheter inlet tubing.
